(19) United States
(12) Patent Application Publication
(10) Pub. No.: US 2015/0266964 A1
(43) Pub. Date: Sep. 24, 2015

(54) METHODS FOR ACCELERATING IMMUNE REGENERATION

(71) Applicants: REGENTS OF THE UNIVERSITY OF MINNESOTA, Minneapolis, MN (US); SANFORD-BURNHAM MEDICAL RESEARCH INSTITUTE, La Jolla, CA (US)

(72) Inventors: Bruce R. Blazar, Golden Valley, MN (US); Heather E. Stefanski, Mendota Heights, MN (US); Carl F. Ware, La Jolla, CA (US)

(21) Appl. No.: 14/433,145

(22) PCT Filed: Oct. 4, 2013
(86) PCT No.: PCT/US2013/063463

§ 371 Date: Apr. 2, 2015

Related U.S. Application Data
(60) Provisional application No. 61/709,584, filed on Oct. 4, 2012.

(51) Int. Cl. C07K 16/28

(52) U.S. Cl. CPC C07K 16/2878; C07K 2317/75

(57) ABSTRACT

This disclosure describes methods that generally include administering to an immune compromised subject an amount of a lymphotoxin β receptor (LTβR) agonist effective to increase immune function in the subject compared to a suitable control immune compromised subject. In some cases, the method can result decreasing the period of immune deficiency in the subject compared to a suitable control immune compromised subject.

### CROSS-REFERENCE TO RELATED APPLICATION

This application claims priority to Provisional Patent Application Ser. No. 61/709,584, filed Oct. 4, 2012, which is incorporated herein by reference.

### GOVERNMENT FUNDING

This invention was made with government support under NIH R01 CA72669-15, NIH R01 AI081918-04, and NIH 2P01 CA065493-14 awarded by the National Institutes of Health. The Government has certain rights in the invention.

### SUMMARY

This disclosure describes, in one aspect, a method of accelerating immune regeneration. Generally, the method includes administering to an immune compromised subject an amount of a lymphotoxin β receptor (LTβR) agonist effective to increase immune function in the subject compared to a suitable control immune compromised subject.

In another aspect, this disclosure describes a method of improving bone marrow transplant therapy. Generally, this method includes administering to a bone marrow transplant recipient an amount of a lymphotoxin β receptor (LTβR) agonist effective to increase immune function in the bone marrow recipient.

In another aspect, this disclosure describes a method that generally includes administering to a bone marrow transplant recipient an amount of a lymphotoxin β receptor (LTβR) agonist effective to increase immune function in the bone marrow recipient compared to a suitable control bone marrow recipient.

In yet another aspect, this disclosure describes a method for decreasing a period of immune deficiency. This method generally includes administering to an immune compromised subject an amount of a lymphotoxin β receptor (LTβR) agonist effective to decrease the period of immune deficiency in the subject compared to a suitable control immune compromised subject.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Experimental design.

FIG. 2. αLTβR restores lymph node architecture and CCL21 expression. αLTβR mAb (d 28-40) restores lymph node size with well-developed T-cell and B-cell zones, and CCL21 expression on d50 in congenic bone marrow transplant mice.

FIG. 3. αLTβR mAb restores spleen architecture, T-cell and B-cell demarcation and CCL21 (upper) and CXCL13 (middle, lower) expression. Expression of B220, CD3, and chemokine are shown.

FIG. 4. αLTβR mAb given to congenic or allogeneic bone marrow transplant recipients results in a striking improvement in VSV-ova clearance; PFU, plaque-forming units. ***p<0.001

### DETAILED DESCRIPTION OF ILLUSTRATIVE EMBODIMENTS

This disclosure describe a method that generally includes administering to an immune compromized subject an amount of a LTβR agonist effective to increase immune function in the subject compared to a suitable control immune compromised subject and/or decrease the period of immune deficiency in the subject compared to a suitable control immune compromised subject.

As used herein, the following terms shall have the indicated meaning.

“LTβ” refers to lymphotoxin β; “LTβR” refers to lymphotoxin β receptor.

“BMT” refers to bone marrow transplant.

Bone marrow stem cell transplantation can be a life-rescuing therapy for patients with one of a variety of conditions such as, for example, cancer and/or autoimmune disease. Bone marrow stem cell transplantation typically involves chemotherapy and/or conditioning of the patient, each of which can compromise the patient's immune defenses for a prolonged period. We demonstrate that agonists of the lymphotoxin β receptor (LTβR) pathway can help to functionally reconstitute the immune system of bone marrow transplant patients. Bone marrow transplant patients who are treated with a LTβR agonist can exhibit a reduced risk of cancer, infections, and/or autoimmune diseases compared to bone marrow transplant patients who are not treated with a LTβR.

Bone marrow stem cell transplantation can be a life saving option for patients having a malignant or non-malignant disease such as, for example, leukemia, lymphoma, multiple myeloma, aplastic anemia, metabolic diseases or other related cancers. However, patients can remain immune-compromised for a or more following a transplant procedure, depending on factors such as, for example, infection, graft versus host disease (GVHD), and/or immunosuppressive drugs. Bone marrow transplant recipients frequently suffer from infections that jeopardize successful engraftment. For example, cytomegalovirus (CMV, a βherpesvirus) infection can occur in 40%-70% of bone marrow transplant patients and can contribute to graft failure, GVHD, and/or superimposed fungal and/or bacterial infections. Despite efforts to prevent CMV infection with pre-emptive or prophylactic use of the antiviral ganciclovir, CMV disease as end organ damage still can occur in approximately 10% of patients and can carry a mortality rate of 30%-90%.

Thus, there is a clear need to intervene with drugs and/or methods that can decrease the time and extent of immune deficiency following bone marrow transplantation. Accelerating the time to immune reconstitution may significantly increase the rate of successful engraftment and decrease in-patient costs.

Delayed immune reconstitution in bone marrow transplant recipients may result from damage to peripheral lymphoid tissues such as, for example, lymph nodes and Peyer's patches. Chemotherapy and conditioning regimes used prior to transplantation can damage stromal cells and/or deplete lymphocytes, which can disrupt the architecture of the lymphoid organs and suppress immune function. Newly generated lymphocytes arising in the bone marrow and thymus typically cannot repopulate, organize, and mount immune responses in the damaged peripheral lymphoid tissues. Lymphocyte-stromal cell communication through the lymphotoxin-β receptor (LTβR) pathway is involved in establishing lymphoid tissue architecture and immune function. Thus, re-establishing LTβR signaling through therapeutic intervention can accelerate engraftment and decrease the likelihood, severity, and/or extent of graft-threatening infections.

We demonstrate that administering a LTβR agonist to immune-compromised patients can decrease the time necessary to regenerate immune function. For example, in patients receiving a bone marrow transplant, stimulation of the LTβR can accelerate regeneration of lymphoid tissue architecture and/or promote engraftment of lymph nodes, thereby promoting restoration of immune function, which can decrease the risk that the patient will develop an infectious disease and/or cancer.

Agonists of the LTβR that can promote LTβR signalling include, for example, multivalent antibodies (, an agonistic anti-LTβR antibody) or natural or engineered soluble ligands (, LTαβ and LIGHT). These LTβR agonists may be clinicaly useful for treating patients that have undergone immune suppressive therapy and/or conditioning therapy associated with syngeneic or allogeneic organ transplantation.

LTβR signaling can activate responses controlling growth, differentiation, and death of cells involved in dendritic cell (DC) homeostasis, interferon responses to pathogens, and/or the formation and organization of peripheral lymphoid organs. Lymphoid-tissue architecture involves homeostatic signaling to maintain its basal functional state. Blocking LTβR signaling can reduce lymph node cellularity and can impair lymphocyte entry due to decreased levels of peripheral lymph node addressin and the mucosal addressin, Mad-CAM, on high endothelial venules (Browning et al. Immunity 23, 539-550 (2005)). LTβR binds LIGHT (also known as tumor necrosis factor ligand superfamily member 14, TNFSF14, and CD258) and LTαβ heterotrimers (LTα1β2; LTα2β1), LIGHT also binds the herpes virus entry mediator and LTαβ binds TNFRI and TNFRII (Norris & Ware, Adv Exp Med Biol 597, 160-172 (2007)).

LTβR signaling operates in part via recruitment of TNFR activating factors-2, -3, or -5 and activating NFκB (Norris & Ware, Adv Exp Med Biol 597, 160-172 (2007)). An NFκB-inducing kinase mutation results in alymphoplasia in mice, resulting in phenotypes similar to LTβR-/-mice. Alymphoplastic mice lack all lymph nodes, Peyer's patches, and most lymphoid tissues. Bone marrow recipients with alymphoplasia can experience significantly less severe acute graft versus host disease, with the lowest severity being in the alymphoplasia, splenectomized recipients (Anderson et al. Blood 111, 5242-5251 (2008)). Thus, secondary lymphoid organs are involved in supporting T cell localization, activation, and/or access to lymph node signals necessary for T cell survival.

Potential targets activated by the LTαβ-LTβR-NEκB signaling pathway include, for example, CXCL13, CCL19 and CCL21 and their receptors CXCR5 and CCR7. Indeed, CXCR5-/- and CCR7-/-mice have defective lymphoid organogenesis. Consistent with LTβR signaling being involved in lymphoid organogenesis, LTα-/-mice, LTβ-/-mice, and LTβR-/-mice often exhibit a deficiency in lymph node formation. These mice exhibit a deficiency in lymphoid tissue inducer cells, which are involved in fetal lymphoid organogenesis and adult lymph node regeneration. The observed deficiency may be a result of LTβR+ fibroblastic reticular cells failing to receive signals for their regeneration and/or induction of CXCL13, CCL19, and CCL21.

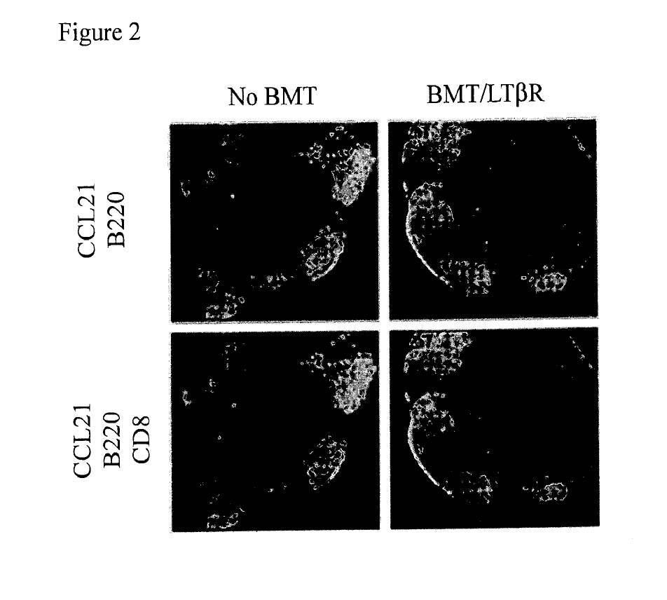

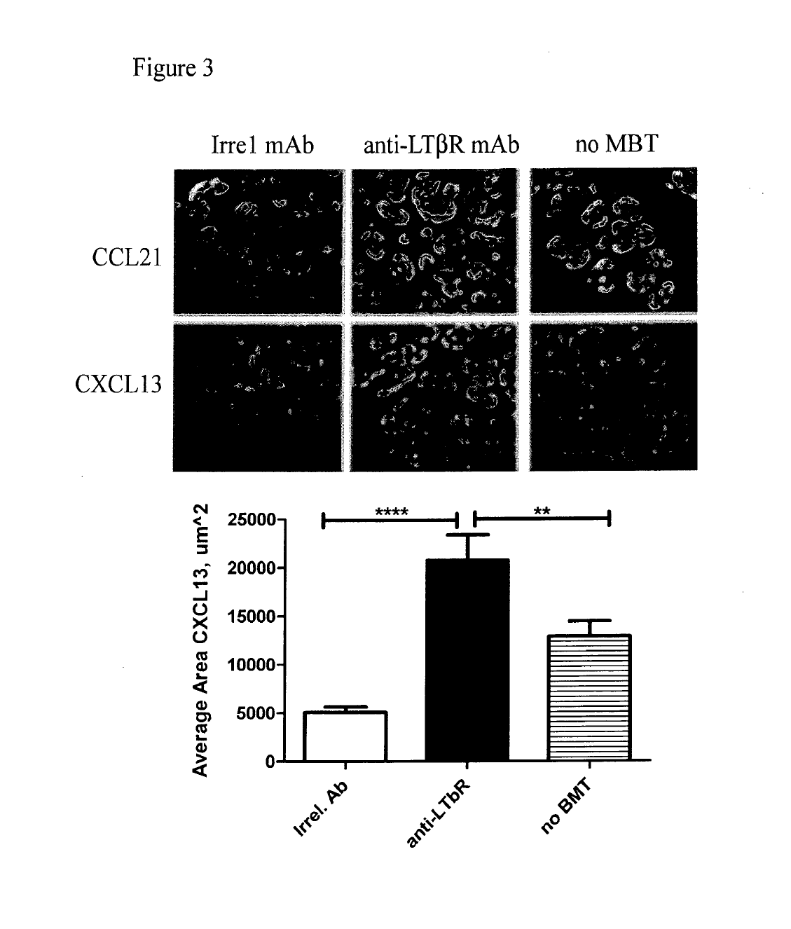

In addition to quantifying LTα1β2, LTα2β1, and LTβR signals within the lymph node, we used gain of function to determine how LTβR signals influence the severity of lymph node injury, lymph node injury repair, and immune reconstitution and function following bone marrow transplant. Agonistic anti-LTβR mAb (4H8¹⁰) given to lethally irradiated congenic bone marrow transplant recipients every three days from 28-40 days post-bone marrow transplant restores CCL21, restores lymph node size and architecture, and results in well-developed T-cell and B-cell zones (FIG. 2) compared to the highly defective lymph node in bone marrow transplant controls (not shown). FIG. 3 illustrates that the architectural, T-cell and B-cell demarcation, and chemokine expression augmented by anti-LTβR mAb is not restricted to lymph nodes.

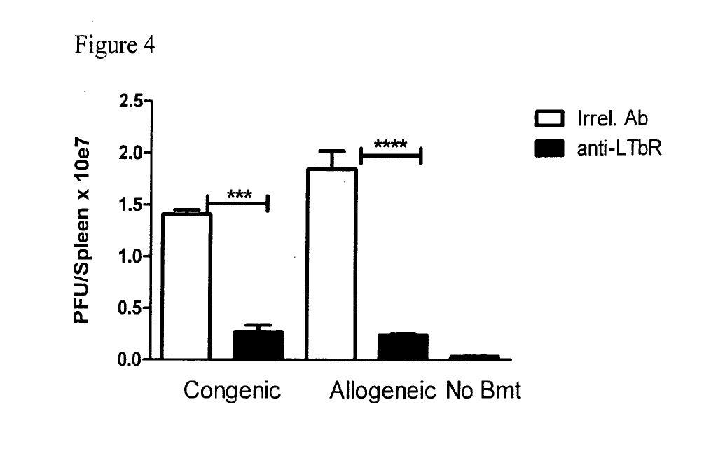

We also tested the ability of the mice to respond to the viral pathogen, VSV-OVA. In order to do so, irradiated B6 mice were given a bone marrow transplant with either congenic (B6-CD45.2) or allogeneic (BALBc:H2d) cells, then were given either anti-LTβR or irrelevant mAb (d.14-35) before being challenged with VSV-OVA on day 42 post-transplant. VSV-OVA clearance (d.43) was markedly increased by anti-LTβR mAb in both congenic and allogeneic bone marrow transplant recipients (FIG. 4). Compared to irrelevant mAb, αLTβR mAb in congenic or allogeneic recipients resulted in a 5-fold to 11-fold increase in naive CD4+ T cells and greater than a 3-fold increase in CD8+ T cells. Moreover, lymphoid tissue inducer cells were increased 2.4-fold in congenic recipients. Taken together, these results suggest that the agonist mAb to the LTβR could provide the correct signals to improve secondary lymphoid tissue organization and recruitment of lymphocytes.

Thus, in the process of developing new therapies for and exploring the mechanisms that contribute to acute myeloid leukemia (AML) relapse following bone marrow transplant, we discovered that the bone marrow transplant procedure itself contributed to the failure to AML relapse. We discovered that radiation-induced conditioning injured host secondary lymphoid organs. For example, we observed small lymph nodes and disorganized microarchitecture³, low numbers of recent thymic emigrants, low numbers of endogenously generated T cells that localized to lymph nodes, diminished expression of T-cell and B-cell chemokines (CCL19, CCL21, CXCL13), and lymph node stromal cells depletion (Kelly et al. Blood 115, 1088-1097 (2010)).

We discovered that administering an agonistic anti-lymphotoxin receptor antibody to bone marrow transplant recipients mediated restoration of CCL21 expression (associated with improved lymph node architecture), increased lymph node size and T cell content, and a significant augmentation in the endogenous immune response to pathogen challenge (Listeria monocytogenes), virus (Vesicular stomatitis virus), or tumor cells (AML).

Thus, we have discovered a novel fundamental defect in the stromal microenvironment of lymphoid tissues that contributes to poor immune function following bone marrow transplant. Regenerating the lymphoid compartment can increase endogenous donor T cell recovery following bone marrow transplant and may improve the efficacy of adoptively transferred T cells used to treat relapse. Relapse following bone marrow transplant can occur, for example, in more aged bone marrow transplant recipient (McClune et al. J Clin Oncol 28, 1878-1887 (2010)) and in patients with known lymph node injury (e.g., HIV patients with lymph node fibrosis; Zeng et al. J Clin Invest 121, 998-1008 (2011)). Since these therapies are readily translatable, our studies have the potential to change the practice of bone marrow transplantation.

While described above in the context of an exemplary embodiment in which the immune compromised subject is a bone marrow transplant recipient, the methods described herein can provide treatment for a subject that is immune compromised for any reason including either primary immunodeficiencies or immunodeficiencies that are secondary to another condition or a treatment regimen for another condition. Exemplary primary immunodeficiencies include, for example, humoral immune deficiencies, T cell deficiencies, neutropenia, asplenia, and/or complement deficiencies. Exemplary conditions that can elicit a secondary immunodeficiency include, for example, malnutrition, cancers (especially those of the blood and/or bone marrow), and certain chronic infections (e.g., HIV). Exemplary treatments that can elicit a secondary immunodeficiency include, for example, chemotherapy, certain antirheumatic drugs (e.g., disease-modifying antirheumatic drugs (DMARDs) such as, for example, adalimumab, etanercept, infliximab, rituximab, and methotrexate), immunosuppressive drugs (e.g., Cyclosporin A, tacrolimus, sirolimus) and glucocorticoids (e.g., prednisone).

We describe, therefore, a method that generally includes administering to an immune compromized subject an amount of a LTβR agonist effective to increase immune function in the subject compared to a suitable control immune compromised subject and/or decrease the period of immune deficiency in the subject compared to a suitable control immune compromised subject.

LTβR agonist can be any suitable agonist of lymphotoxin β receptor including, for example, an agonist anti-LTβR antibody (e.g., 4H8¹⁰. There are a number of LTβR antagonists (LTβR-Fc) and knockout mice that have shown the importance of LTβR signaling including LTα-/-mice, LTβ-/-mice, and LTβR-/-mice.

The LTβR agonist may be formulated in a composition along with a “carrier.” As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the LTOR agonist, its use in the therapeutic compositions is contemplated. Supplementary active ingredients also can be incorporated into the compositions.

By “pharmaceutically acceptable” is meant a material that is not biologically or otherwise undesirable, i.e., the material may be administered to an individual along with the LTR agonist without causing any undesirable biological effects or interacting in a deleterious manner with any of the other components of the pharmaceutical composition in which it is contained.

An LTβR agonist may be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). It is foreseen that a composition can be administered to a mucosal surface, such as by administration to, for example, the nasal or respiratory mucosa (e.g., by spray or aerosol). A composition also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing the LTβR agonist into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

An LTβR agonist may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The foimulation may further include one or more additives including such as, for example, an adjuvant, a skin penetration enhancer, a colorant, a fragrance, a flavoring, a moisturizer, a thickener, and the like.

Thus, in another aspect, this disclosure describes the use of an LTβR agonist in the manufacture of a pharmaceutical composition effective to, for example, accelerate immune regeneration in a subject in need of immune regeneration.

Referring back to the methods that involve administering an LTβR agonist to a subject, the amount of LTβR agonist administered can vary depending on various factors including, but not limited to, the specific LTβR agonist, the weight, physical condition, and/or age of the subject, and/or the route of administration. Thus, the absolute weight of LTβR agonist included in a given unit dosage form can vary widely, and depends upon factors such as the species, age, weight and physical condition of the subject, as well as the method of administration. Accordingly, it is not practical to set forth generally the amount that constitutes an amount of LTβR agonist effective for all possible applications. Those of ordinary skill in the art, however, can readily determine the appropriate amount with due consideration of such factors.

In some embodiments, the method can include administering sufficient LTβR agonist to provide a dose of, for example, from about 100 ng/kg to about 50 mg/kg to the subject, although in some embodiments the methods may be performed by administering FOR agonist in a dose outside this range. In some of these embodiments, the method includes administering sufficient LTβR agonist to provide a dose of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt/kg^{0.425}×height cm^{0.725})×0.007184.

In some embodiments, the method can include administering sufficient LTβR agonist to provide a dose of, for example, from about 0.01 mg/m² to about 10 mg/m².

In some embodiments, the LTβR agonist may be administered, for example, from a single dose to multiple doses per week, although in some embodiments the method can be performed by administering the LTβR agonist at a frequency outside this range. In certain embodiments, the LTβR agonist may be administered from about once per month to about five times per week. In certain embodiments, the LTβR agonist may be administered three times per week.

In some embodiments, the LTβR therapy can begin after an event that results in compromised immunity. This could start as early as Day 1 post-transplant to years post-transplant. For example, a patient that has chronic graft versus host disease can be immunocompromised for years after receiving the bone marrow transplant. In these cases, therapy can be initiated many years after the event that results in compromised immunity.

In some embodiments, one can administer LTβR for a period of at least a few weeks, for up to months depending on the response to the immune system.

### EXEMPLARY EMBODIMENTS

### Embodiment 1

A method of accelerating immune regeneration, the method comprising:

administering to an immune compromised subject an amount of a lymphotoxin receptor (LTβR) agonist effective to increase immune function in the subject compared to a suitable control immune compromised subject.

### Embodiment 2

A method of improving bone marrow transplant therapy, the method comprising:

administering to a bone marrow transplant recipient an amount of a lymphotoxin β receptor (LTβR) agonist effective to increase immune function in the bone marrow recipient.

### Embodiment 3

A method comprising: administering to a bone marrow transplant recipient an amount of a lymphotoxin β receptor (LTβR) agonist effective to increase immune function in the bone marrow recipient compared to a suitable control bone marrow recipient.

### Embodiment 4

The method of any preceding Embodiment wherein the increase in immune function comprises an increase in lymph node size compared to a suitable control at the same time post-bone marrow transplant.

### Embodiment 5

The method of any preceding Embodiment wherein the increase in immune function comprises restoring spleen architecture.

### Embodiment 6

The method of Embodiment 5 wherein restoring spleen architecture comprises increased demarcation between T cell zones and B cell zones in the spleen compared to a suitable control at the same time post-bone marrow transplant.

### Embodiment 7

The method of any preceding Embodiment wherein the increase in immune function comprises an increase in expression of CXCL13, CCL19, or CCL21 compared to a suitable control at the same time post-bone marrow transplant.

### Embodiment 8

A method for decreasing a period of immune deficiency, the method comprising:

administering to an immune compromised subject an amount of a lymphotoxin β receptor (LTβR) agonist effective to decrease the period of immune deficiency in the subject compared to a suitable control immune compromised subject.

### Embodiment 9

The method of Embodiment 8 wherein the decrease the period of immune deficiency comprises an increase in lymph node size compared to a suitable control at the same time post-bone marrow transplant.

### Embodiment 10

The method of Embodiment 8 or Embodiment 9 wherein the decrease in immune deficiency comprises restoring spleen architecture.

### Embodiment 11

The method of Embodiment 10 wherein restoring spleen architecture comprises increased demarcation between T cell zones and B cell zones in the spleen compared to a suitable control at the same time post-bone marrow transplant.

### Embodiment 12

The method of any one of Embodiments 8-11 wherein the decrease in immune deficiency comprises an increase in expression of CXCL13, CCL19, or CCL21 compared to a suitable control at the same time post-bone marrow transplant.

### Embodiment 13

The method of any preceding Embodiment wherein the LTβR agonist comprises an agonist anti-LTβR antibody.

### Embodiment 14

The method of any preceding Embodiment wherein the LTβR agonist is administered after an event that results in compromised immunity.

### Embodiment 15

The use of a LTβR agonist in the manufacture of a pharmaceutical composition effective for accelerating immune regeneration in a subject.

### Embodiment 16

The use of a LTβR agonist in the manufacture of a pharmaceutical composition effective for improving bone marrow transplant therapy in a subject.

### Embodiment 17

The use of Embodiment 15 or Embodiment 16 wherein the pharmaceutical composition increases immune function when administered to the subject.

### Embodiment 18

The use of Embodiment 17 wherein the increase in immune function comprises an increase in lymph node size compared to a suitable control at the same time post-bone marrow transplant.

### Embodiment 19

The use of Embodiment 17 wherein the increase in immune function comprises restoring spleen architecture.

### Embodiment 20

The use of Embodiment 17 wherein restoring spleen architecture comprises increased demarcation between T cell zones and B cell zones in the spleen compared to a suitable control at the same time post-bone marrow transplant.

### Embodiment 21

The use of any one of Embodiments 17-20 wherein the increase in immune function comprises an increase in expression of CXCL13, CCL19, or CCL21 compared to a suitable control at the same time post-bone marrow transplant.

### Embodiment 22

The use of a LTβR agonist in the manufacture of a pharmaceutical composition effective for decreasing a period of immune deficiency.

### Embodiment 23

The use of Embodiment 22 wherein the decrease the period of immune deficiency comprises an increase in lymph node size compared to a suitable control at the same time post-bone marrow transplant.

### Embodiment 24

The use of Embodiment 22 or Embodiment 23 wherein the decrease in immune deficiency comprises restoring spleen architecture.

### Embodiment 25

The use of Embodiment 24 wherein restoring spleen architecture comprises increased demarcation between T cell zones and B cell zones in the spleen compared to a suitable control at the same time post-bone marrow transplant.

### Embodiment 26

The use of any one of Embodiments 22-25 wherein the decrease in immune deficiency comprises an increase in expression of CXCL13, CCL19, or CCL21 compared to a suitable control at the same time post-bone marrow transplant.

### Embodiment 27

The use of any one of Embodiments 15-26 wherein the LTβR agonist comprises an agonist anti-LTβR antibody.

### Embodiment 28

The use of any one of Embodiments 15-27 wherein the LTβR agonist is administered after an event that results in compromised immunity.

As used in herein, the term “and/or” means one or all of the listed elements or a combination of any two or more of the listed elements; the turns “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

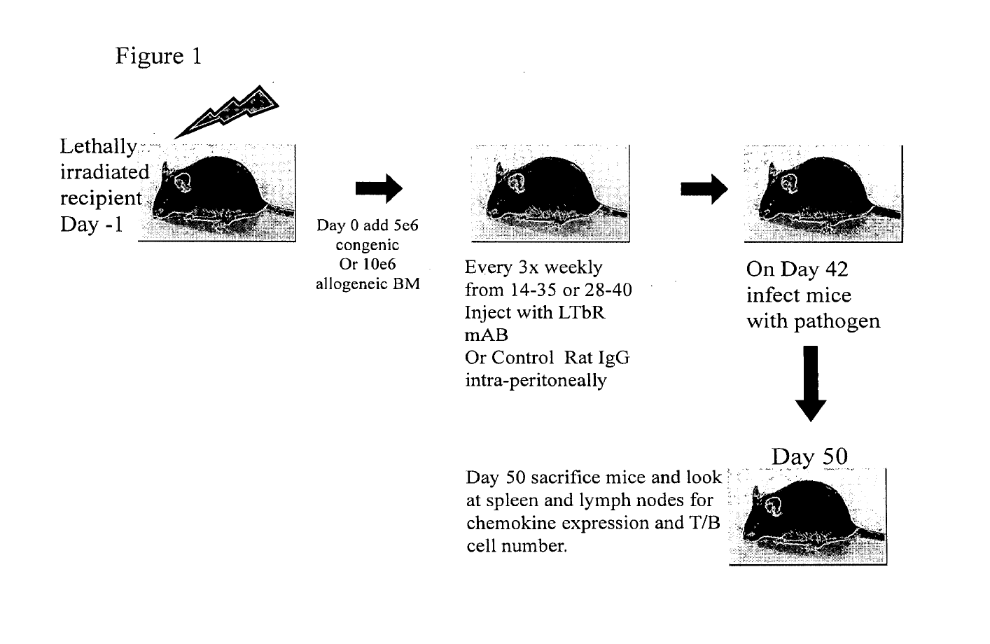

FIG. 1 summarizes the following experimental procedure. C57BL6-CD45.1 (B6, H2b) mice were subjected to lethal irradiation (1100 Gy). The next day (Day 0), the irradiated mice were treated with T cell depleted bone marrow that is either congenic (5×10⁶ B6-CD45.2, H2b cells) or allogeneic (10⁶ BALBc:H2d cells). The BMT recipient mice were treated with 100 μg of either agonistic anti-LTβR mAb 4H8¹⁰ or a control rat IgG (Rat IgG, Rockland Immunochemicals, Inc., Gilbertsville, Pa.) three times per week, from Day 28 through Day 40.

On Day 42, mice were inoculated with recombinant Listeria monocytogenes strain ΔactA-Lm-OVA expressing full-length chicken ovalbumin (OVA) bacteria in early logarithmicphase grown in brain heart infusion (BHI) broth at 37° C. Congenic BM transplant recipients were infected with 10⁶ colony-forming units (CFUs) of ΔactA-Lm-OVA. Eight days after infection, livers were homogenized in 0.05% Triton X-100PBS, plated onto BHI plates, and Listeria monocytogenes colonies were enumerated after 24 hours at 37° C.

On Day 50, the mice were sacrificed and the lymph nodes examined histologically to assess lymph node architecture. Tissues were embedded in OCT embedding compound and snap-frozen in liquid nitrogen. For LN/spleen analysis, 6-μm cryosections were acetone-fixed and stained for CCL21 (R&D Systems, Inc., Minneapolis, Minn.) along with B220-FITC (clone RA3-6B2; BD) and Rat anti-mouse CD8a Cy5 (eBioscience, Inc., San Diego, Calif.) for 3 hours at room temperature. CCL21 signal was amplified with Tyramide Signal Amplification kit according to the manufacturer's instructions (Invitrogen, Life Technologies Corp., Grand Island, N.Y.). Slides were mounted with VECTASHIELD (Vector Laboratories, Inc., Burlingame, Calif.) and images were acquired through a 10×/0.40 Olympus UPlanApo or 40×/0.80 Olympus UPlanApo Oil lens and an Olympus FV500 camera, compiled with Fluoview software (v.4.3), then analyzed and cropped in Adobe Photoshop CS2. Results are shown in FIG. 2 and FIG. 3.

### Example 2

Mice were irradiated and given bone marrow transplants as described in Example 1. On Day 42, the mice were infected with 10⁶ plaque forming units of vesicular stomatitis virus (VSV-ova) (Kim et al., 1998, Proc. Natl. Acad. Sci., 95:10814-10819). On Day 43, the mice were sacrificed and analyzed as described in Example 1.

Results are shown in FIG. 4.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method of accelerating immune regeneration, the method comprising:
administering to an immune compromised subject an amount of a lymphotoxin β receptor (LTβR) agonist effective to increase immune function in the subject compared to a suitable control immune compromised subject.

2. A method of improving bone marrow transplant therapy, the method comprising:
administering to a bone marrow transplant recipient an amount of a lymphotoxin β receptor (LTβR) agonist effective to increase immune function in the bone marrow recipient.

3. A method comprising:
administering to a bone marrow transplant recipient an amount of a lymphotoxin β receptor (LTβR) agonist effective to increase immune function in the bone marrow recipient compared to a suitable control bone marrow recipient.

4. The method of claim 1 wherein the increase in immune function comprises an increase in lymph node size compared to a suitable control at the same time post-bone marrow transplant.

5. The method of claim 1 wherein the increase in immune function comprises restoring spleen architecture.

6. (canceled)

7. The method of claim 1 wherein the increase in immune function comprises an increase in expression of CXCL13, CCL19, or CCL21 compared to a suitable control at the same time post-bone marrow transplant.

8. A method for decreasing a period of immune deficiency, the method comprising:
administering to an immune compromised subject an amount of a lymphotoxin 0 receptor (LTβR) agonist effective to decrease the period of immune deficiency in the subject compared to a suitable control immune compromised subject.

9. The method of claim 8 wherein the decrease the period of immune deficiency comprises an increase in lymph node size compared to a suitable control at the same time post-bone marrow transplant.

10. The method of claim 8 wherein the decrease in immune deficiency comprises restoring spleen architecture.

11. The method of claim 10 wherein restoring spleen architecture comprises increased demarcation between T cell zones and B cell zones in the spleen compared to a suitable control at the same time post-bone marrow transplant.

12. The method of claim 8 wherein the decrease in immune deficiency comprises an increase in expression of CXCL13, CCL19, or CCL21 compared to a suitable control at the same time post-bone marrow transplant.

13. The method of claim 1 wherein the LTβR agonist comprises an agonist anti-LTβR antibody.

14. The method of claim 1 wherein the LTβR agonist is administered after an event that results in compromised immunity.

15-28. (canceled)

29. The method of claim 2 wherein the increase in immune function comprises an increase in lymph node size compared to a suitable control at the same time post-bone marrow transplant.

30. The method of claim 2 wherein the increase in immune function comprises restoring spleen architecture.

31. The method of claim 2 wherein the increase in immune function comprises an increase in expression of CXCL13, CCL19, or CCL21 compared to a suitable control at the same time post-bone marrow transplant.

32. The method of claim 2 wherein the LTβR agonist comprises an agonist anti-LTβR antibody.

33. The method of claim 2 wherein the LTβR agonist is administered after an event that results in compromised immunity.

34. The method of claim 3 wherein the increase in immune function comprises an increase in lymph node size compared to a suitable control at the same time post-bone marrow transplant.

35. The method of claim 3 wherein the increase in immune function comprises restoring spleen architecture.

36. The method of claim 3 wherein the increase in immune function comprises an increase in expression of CXCL13, CCL19, or CCL21 compared to a suitable control at the same time post-bone marrow transplant.

37. The method of claim 3 wherein the LTβR agonist comprises an agonist anti-LTβR antibody.

38. The method of claim 3 wherein the LTβR agonist is administered after an event that results in compromised immunity.

39. The method of claim 8 wherein the LTβR agonist comprises an agonist anti-LTβR antibody.

40. The method of claim 8 wherein the LTβR agonist is administered after an event that results in compromised immunity.
